(19) United States
(12) Patent Application Publication
(10) Pub. No.: US 2022/0279830 A1
(43) Pub. Date: Sep. 8, 2022

(54) FOOD COMPOSITION

(71) Applicant: Brauus LLC, Miami, FL (US)

(72) Inventors: Cátia COELHO, Darien, CT (US); Rodolfo COELHO, Darien, CT (US)

(21) Appl. No.: 17/625,394

(22) PCT Filed: Jun. 30, 2020
(86) PCT No.: PCT/US20/40243

§ 371 Date: Jan. 7, 2022

Related U.S. Application Data
(60) Provisional application No. 62/871,945, filed on Jul. 9, 2019.

(51) Int. Cl. A23L 33/105; A23L 33/21; A23L 33/125; A23L 33/115; A23L 19/00; A61K 31/733

(52) U.S. Cl. CPC A23L 33/105; A23L 33/21; A23L 33/125; A23L 33/115; A23L 19/09; A61K 31/733; A23V 2002/00

(57) ABSTRACT

A food composition containing green banana and inulin and having a low glycemic index and/or a low glycemic load is described. The food composition is delicious and healthy, contains natural, whole ingredients, high amounts of dietary fiber, healthy fats, and no added sugar. Such a composition satisfies the desire for a sweet treat while nurturing the body.

### CROSS-REFERENCE TO RELATED APPLICATION

This application claims priority to U.S. provisional application No. 62/871,945, filed Jul. 9, 2019, the disclosure of which is herein incorporated by reference in its entirety.

### BACKGROUND OF THE INVENTION

There are approximately 40 trillion bacteria in the body of an individual, mostly in the intestines. These bacteria are collectively known as gut microbiome or microbiota. Gut microbiome is very important for the body's health and is nourished by various foods, particularly foods which are rich in dietary fiber.

For example, foods including resistant starch promote the nourishment of gut microbiome. Resistant starch functions similar to soluble fiber and is resistant to digestion in the upper gastrointestinal tract. More particularly, resistant starch passes through the small intestine undigested and travels to the large intestine where it feeds healthy gut bacteria. The healthy gut bacteria break down or digest the resistant starch and are therefore fueled by the resistant starch. As the healthy bacteria break down the resistant starch, they form compounds, including gases and short-chain fatty acids, such as butyrate. Butyrate is absorbed by the large intestine and has numerous beneficial effects.

Fructans are another category of non-digestible food ingredients that nourish and stimulate the growth of the beneficial microbiome of the large intestine. Because fructans cannot be digested by human digestive enzymes, they travel to the lower gastrointestinal tract where they are digested/fermented by beneficial bacteria. As a result, short-chain fatty acids (e.g., butyrate) are produced.

There is scientific evidence that resistant starch and fructans, which are both types of prebiotic fibers, have a number of beneficial health effects, including reducing the risk for certain types of cancer, enhancing calcium absorption, improving bowel function, improving insulin sensitivity, lowering blood sugar levels, increasing satiety, regulating anxiety, cognition, mood and pain, and the like.

Glycemic index (GI) is a term which is used in the food industry to classify and relatively rank carbohydrates in foods according to how they affect blood glucose levels. GI is a quantitative measure of how quickly (and how much) a food will cause blood sugar levels to rise. The glycemic index is scaled from 0-100 with 100 being the index for glucose. Foods with a lower glycemic index are digested and absorbed more slowly, causing smaller and less rapid increases in your blood sugar. These foods will typically have more fiber and/or protein. Meanwhile, foods with higher glycemic indices are rapidly absorbed and have larger and more immediate impact on your blood sugar. These foods tend to have more rapidly absorbed carbohydrates and are often more processed. Carbohydrates with low GI values (55 or less) are more slowly digested, absorbed, and metabolized and cause a lower and slower rise in blood glucose and, therefore, usually insulin levels as well.

Glycemic load (GL) is the translation of the Glycemic Index into practical terms. It is calculated by multiplying the amount of digestible carbs in a serving of food by the Glycemic Index of that specific food. In other words, it is a quantitative measure of how much a specific portion of food will impact someone's blood sugar curve. The glycemic load measures both the quality and the quantity of carbs in foods, making it a good way to guide daily decisions regarding food. The GL of a food estimates how much the food will raise a person's blood glucose level after eating it; one unit of glycemic load approximates the effect of eating one gram of glucose. GL takes into account the planned portion size of food and the GI of the particular food. Accordingly, foods having low GI values (55 or less) and low GL values (10 or less) are desirable because they may help in weight maintenance, weight loss, blood sugar level control, and may be protective against diseases such as diabetes, cancers, and heart disease.

Therefore, it would be desirable to provide a food composition containing ingredients which promote the health of the body's gut microbiome. It would also be desirable, particularly for those suffering with obesity, diabetes, or pre-diabetes, to provide such food compositions with low glycemic index and/or low glycemic load.

### BRIEF SUMMARY OF THE INVENTION

A composition according to one embodiment of the disclosure contains green banana and inulin and has a low glycemic index and/or a low glycemic load.

### DETAILED DESCRIPTION OF THE INVENTION

The disclosure relates to food compositions which promote gut microbiome nourishment. More particularly, the disclosure relates to novel nutritional food compositions comprising a combination of one or more resistant starches and one or more types of fructans. In a preferred embodiment, the food composition comprises a combination of green banana as the source of resistant starch and inulin as the fructan.

In one embodiment, the green banana comprises a green banana biomass. The green banana biomass may be formed by cooking, peeling and shearing the pulp of a plurality of green bananas so as to form a green banana puree.

Other appropriate forms of the green banana include, without limitation, raw green banana and green banana flour. It will also be understood that the green banana component of the food composition may comprise green banana biomass, raw green banana and/or green banana flour, either alone or in combination with each other. The food composition comprises green banana in an amount of about 5% to about 70% by weight, more preferably about 10% to about 60% by weight, most preferably about 10% to about 25%. It will be understood by those skilled in the art that the specific concentration of the green banana may vary depending upon various factors of the desired food product, such as nutritional content, taste, texture and the like.

The inulin is preferably initially provided in powder form and is preferably derived from blue agave. Inulin is a plant-based prebiotic, soluble fiber, produced by many fruits and vegetables as a source of energy. Inulin helps to maintain healthy digestion and decrease the risk of disease, as well as being naturally sweet. The food composition preferably comprises inulin in an amount of about 3% to 45%, and more preferably about 7% to 20% by weight. It will be understood by those skilled in the art that the specific concentration of the inulin may vary depending upon various factors of the desired food product, such as nutritional content, taste, texture and the like.

The food composition preferably has a low glycemic index and/or a low glycemic load. For the purposes of this disclosure, the term “low glycemic index” refers to a GI of 55 or less; GI values of 56 to 69 are “medium” and GI values of 70 or higher are “high.” The glycemic load referred to herein is calculated as GL=GI×g/100, where GI is the glycemic index of the food composition and g is the glycemic carbohydrate content per serving of the food composition. The term “low glycemic load” refers to a GL of 10 or less; GL values of 11 to 20 are “medium” and those having GL of greater than 20 are “high.”

In one embodiment, to facilitate adequate mixing of the inulin with the green banana and further lower the glycemic index and consequent load, the food composition preferably includes a fat component, more preferably a fat component that contains water. The inulin is preferably diluted in or otherwise mixed with the fat component to form an inulin solution which is then mixed with the green banana. Examples of the fat component include, for example, any type of milk, half and half, heavy cream, light cream, coconut milk, almond milk, soy milk, coconut cream, creamed coconut, and any mixture thereof. Most preferably, the inulin is diluted in coconut cream/creamed coconut. The food composition preferably comprises the fat component in an amount of about 15% to 60% by weight, more preferably about 20% to 50% by weight, and most preferably about 30% to about 50% by weight. It will be understood by those skilled in the art that the specific concentration of the fat component may vary depending upon various factors of the desired food product, such as nutritional content, taste, texture and the like.

In one embodiment, the food composition further preferably comprises a sweetening agent, and more preferably a low glycemic sweetening agent such that the food composition would be suitable for consumption by an individual managing diabetes. The food composition preferably does not contain any added sugar. Examples of the sweetening agent include sugar alcohols such as sorbitol, mannitol, xylitol, maltitol, lactitol, or erythritol, monk fruit sweetener, stevia, and allulose, and mixtures thereof; presently preferred is a combination of a sugar alcohol and monk fruit. The total amount of sweetening agent is preferably present in the food composition in an amount of about 2% to 20% by weight, and more preferably about 5% to 15% by weight. It will be understood by those skilled in the art that the specific concentration of the sweetening agent may vary depending upon various factors of the desired food product, such as nutritional content, taste, texture and the like.

In one embodiment, the food composition comprises one or more additives, such as a flavoring agent. For example, the additives included in the food composition may depend on whether the food composition is intended to be sweet or savory. For example, the additive may be a cocoa-based ingredient, a fruit-based ingredient, a nut-based ingredient, a cheese-based ingredient, a spice-based ingredient, a coconut-based ingredient, a vegetable-based ingredient, an herb-based ingredient, a meat-based ingredient, a fish-based ingredient, a soy-based ingredient, a grain-based and the like. In preferred embodiments, the flavoring agents include superfoods, such as antioxidant-rich berries or organic cocoa liquor, and flaxseed. Currently preferred flavors for the food composition include berry, chocolate, and coconut. Additionally, the food composition may include a natural additive such as vanilla. The food composition preferably comprises the additives in a total amount of about 5% to 45% by weight, and more preferably about 10% to 30% by weight, most preferably about 15 to 25% by weight. It will be understood by those skilled in the art that the specific concentration of the additive may vary depending upon various factors of the desired food product, such as nutritional content, taste, texture and the like.

To make the food composition, green banana and inulin, optionally along with one or more of the ingredients discussed above, are mixed together, in the concentrations discussed above, for example using a high speed mixer or similar equipment. In one embodiment, the food composition comprises a mixture of green banana biomass in an amount of about 10% to 20% by weight, inulin in an amount of about 8% to 15% by weight, coconut cream in an amount of about 30% to 50% by weight and a low glycemic sweetener in an amount of about 5% to 15% by weight.

The mixture is then processed and packaged according to food safety best practices to ensure its conformity to industry and regulatory standards such as by heating to an appropriate temperature (such at least about 90° C.) and maintaining the mixture at this temperature for an appropriate time, such as at least about ten minutes. Subsequently, the hot mixture is placed into sterile containers which will serve at the final product containers; the filled containers are immediately sealed, and maintained at an elevated temperature for a predetermined period, then allowed to air cool for a predetermined period. Optionally, the filled containers may also be further subjected to forced cooling. The containers may be made of glass or food grade plastic.

Once packaged, the food composition is shelf-stable at room temperature for up to one year. It may be consumed at room temperature or cold.

The food compositions described herein have many benefits, including being delicious and healthy, containing natural, whole ingredients, high amounts of dietary fiber, no added sugar, and having low glycemic index. Such compositions satisfy the desire for a sweet treat while nurturing the body.

In a preferred embodiment, the food composition contains green bananas, inulin, coconut cream, erythritol, natural flavoring (such as vanilla), monk fruit, and cocoa liquor (for chocolate-flavored composition) or shredded coconut (for coconut-flavored composition). In an alternative preferred embodiment, the food composition contains green bananas, inulin, coconut cream, erythritol, monk fruit, and a mixture of different berries (such as blueberries, raspberries, and maquiberries).

It will be understood that all descriptions of a “% by weight” as included herein refer to a percentage weight of a component or ingredient relative to a total weight of the composition.

The invention will now be described in connection with the following, non-limiting examples.

### Example 1: Preparation of Coconut-Flavored Composition

A coconut-flavored composition was prepared by combining coconut cream, green banana biomass, shredded coconut, inulin, sweetening agents (erythritol and monk fruit extract), and natural flavoring in the amounts shown in Table 1 and mixed using a high speed mixer. The resulting mixture was heated to at least 90° and cooked until the desired consistency was achieved.

TABLE 1
Coconut-flavored Composition

| Component | Weight Percentage Range |
|---|---|
| coconut cream | 40-50 |
| green banana biomass | 10-20 |
| Shredded coconut | 10-20 |
| inulin | 10-20 |
| sweetening agents | 5-15 |
| natural flavoring | 1-10 |

### Example 2: Preparation of Berry-Flavored Composition

A berry-flavored composition was prepared by combining coconut cream, green banana biomass, a mixture of different berries, inulin, sweetening agents (erythritol and monk fruit extract), and natural flavoring in the amounts shown in Table 2 and mixed using a high speed mixer. The resulting mixture was heated to at least 90° and cooked until the desired consistency was achieved.

TABLE 2
Berry-flavored Composition

| Component | Weight Percentage Range |
|---|---|
| coconut cream | 30-40 |
| green banana biomass | 15-25 |
| mixed berries | 10-30 |
| inulin | 10-20 |
| sweetening agents | 5-15 |
| natural flavoring | 1-10 |

### Example 3: Preparation of Chocolate-Flavored Composition

A chocolate-flavored composition was prepared by combining coconut cream, green banana biomass, cocoa liquor, inulin, sweetening agents (erythritol and monk fruit extract), and natural flavoring in the amounts shown in Table 3 and mixed using a high speed mixer. The resulting mixture was heated to at least 90° and cooked until the desired consistency was achieved.

TABLE 3
Chocolate-flavored Composition

| Component | Weight Percentage Range |
|---|---|
| coconut cream | 45-55 |
| green banana biomass | 15-25 |
| cocoa liquor | 5-15 |
| inulin | 5-15 |
| sweetening agents | 5-15 |
| natural flavoring | 1-10 |

### Example 4: Analysis of Coconut-Flavored Composition

The glycemic load of the coconut-flavored composition prepared in Example 1 was determined by measuring the glycemic response after two hours after one serving of the coconut-flavored composition (50 g, served in a bowl with a spoon) and 25 g dextrose control (dissolved in water, consumed twice) were consumed by each of ten subjects on separate days after overnight fasts. For each subject, T/(D/25) was calculated, where T was the glucose AUC (incremental area under the curve) after the test product was eaten and D was the average AUC after the two dextrose controls were consumed. The mean of the resulting values, after excluding outliers (>2SD from the mean) was the EGL (equivalent glycemic load) of the product, and was calculated to be 2±1 (low).

### Example 5: Analysis of Berry- and Chocolate-Flavored Compositions

The glycemic index (GI) of the berry and chocolate-flavored compositions prepared in Examples 2 and 3 were determined using the standard ISO 26642:2010. Specifically, ten subjects consumed each composition (50 g, served in a bowl with a spoon) and 25 g dextrose control (dissolved in water, consumed twice). The GI values are expressed on the glucose scale where the GI of glucose=100 and white bread=71. The glycemic load (GL) of the two compositions was calculated as GI×g/100, where GI is the glycemic index of the composition and g is the glycemic carbohydrate content of one standard serving of the composition (50 g). For the berry-flavored composition, the GI was 27±1 (low) and the GL was 2 (low). For the chocolate-flavored composition, the GI was 27±4 (low) and the GL was 2 (low).

It will be appreciated by those skilled in the art that changes could be made to the embodiment described above without departing from the broad inventive concepts thereof. Also, based on this disclosure, a person of ordinary skill in the art would further recognize that the relative proportions of the components illustrated above could be varied without departing from the spirit and scope of the invention. It is understood, therefore, that this invention is not limited to that particular embodiments disclosed, but it is intended to cover modifications within the spirit and scope of the present invention as defined by the appended claims.

1. A composition comprising green banana, inulin, a low glycemic sweetening agent and a fat component,
wherein the composition has a glycemic index of about 55 or less and/or a glycemic load of about 10 or less,
wherein the sweetening agent is at least one of erythritol and monk fruit, and
wherein the fat component is selected from the group consisting of milk, half and half, heavy cream, light cream, coconut milk, almond milk, soy milk, coconut cream, creamed coconut and mixtures thereof.

2. The composition according to claim 1, wherein the green banana is present in the form of one or more selected from the group consisting of green banana biomass, raw green banana and green banana flour.

3. The composition according to claim 1, wherein the green banana is present in an amount of about 10% to 60% by weight relative to the total weight of the composition and the inulin is present in an amount of about 3% to 45% by weight relative to the total weight of the composition.

4. (canceled)

5. The composition according to claim 1, wherein the sweetening agent is present in an amount of about 5% to 15% by weight relative to a total weight of the composition.

6. The composition according to claim 1, wherein the fat component is present in an amount of about 15% to 60% by weight relative to the total weight of the composition.

7. (canceled)

8. The composition according to claim 1, wherein the composition has a glycemic index of about 30 or less.

9. (canceled)

10. The composition according to claim 1, wherein the composition has a glycemic load of about 2 or less.

11. The composition according to claim 1, wherein the composition has a berry, chocolate, or coconut flavor.

12. (canceled)

13. The composition according to claim 1, wherein the fat component comprises at least one of coconut cream and creamed coconut.
